(19) United States
(12) Patent Application Publication
(10) Pub. No.: US 2002/0009479 A1
(43) Pub. Date: Jan. 24, 2002

(54) Vegetarian foodstuff containing entrapped viable algae

(75) Inventors: Pnina Vardi, Haifa (IL); Konstantin Bloch, Petach Tikva (IL)

Correspondence Address: DR. MARK FRIEDMAN LTD., C/o Bill Polkinghorn, Discovery Dispatch, 9003 Florin Way, Upper Marlboro, MD, 20772, US

(73) Assignee: ALGAL, LTD

(21) Appl. No.: 09/960,363

(22) Filed: Sep. 24, 2001

Related U.S. Application Data
(63) Continuation of application No. 09/406,687, filed on Sep. 27, 1999.

(51) Int. Cl. A61F 9/02

(52) U.S. Cl. 424/437

(57) ABSTRACT

A food composition containing viable algae. The food composition includes primarily entrapped living algae cells capable of photosynthetic multiplication which increases the nutritional content of the food composition. Further disclosed is a food composition including primarily fibrous structures containing living algae cells capable of photosynthetic multiplication within the fibrous structures. Further disclosed are methods for producing the compositions, the methods including the steps of: (a) suspending live algae cells in a suitable medium; (b) placing the suspended live algae cells into a setting bath; and (c) incubating to form entrapped living algae cells. In some cases the method further includes the step of bubbling a gas through the setting bath.

This application is a continuation in part of U.S. Patent application 09/406,687 filed on Sep. 27, 1999.

### FIELD AND BACKGROUND OF THE INVENTION

The present invention relates to a vegetarian foodstuff containing entrapped viable algae and to methods of producing same. The algae is preferably Dunaliella but may further include Chlorella or Spirulina, which belong to the species of unicellular Chlorophyceae. In particular, the invention concerns encapsulated live algae cells in semi-permeable capsules or elongated fibrous structures,

Algae contain various physiological active ingredients. Chlorella, Dunaliella and Spirulina are known to be rich in more than 20 different vitamins, amino acids and minerals, are abundant in beta-carotene and chlorophyll, as well as growth factor. Chlorella, Dunaliella and Spirulina are all non-toxic.

Chlorella, is rich in high quality proteins (50-60% of total mass), carbohydrate (15-20%), fat (10-15%), minerals (6%) and 4% moisture. Chlorella is the one of the highest sources of chlorophyll and also contains Chlorella Growth factor and a higher concentration of Vitamin B12 than beef liver, Chlorella Growth factor has hormone like properties, that has been shown to stimulate tissue repair and promote the growth of children and animals. The synergistic effect of the many nutrients contained in Chlorella help to balance and stabilize functional processes at the cellular level.

Dunaliella algae contain proteins, lipids, sugars and minerals such as iron and calcium. Dunaliella also contains vitamins such as provitamin A and vitamin B group compounds and a variety of other physiologically active ingredients, especially β-carotene.

Chlorella, Spirulina and Dunaliella are used as a health food. They are taken by people and animals in the form of tablets or granules after extraction of the algae in hot water and preparation of a dry powder. Tablets, granules and liquid extracts of Chlorella have been shown to exhibit numerous positive physiological effects. Chlorella stimulates the immune system, displays antioxidant and antitumour activity, exhibits anti-aging properties, has cold preventative action, restores bowel regularity, normalizes bowel flora and stimulates repair of damaged bowels. Chlorella has also been reported to detoxify the body, be useful for post-operative patients, older people, children with growth problems and pregnant women, and is useful in weight gain and weight loss. In addition, Chlorella has been shown to promote solid bone building, healthy gums, teeth and liver. Chlorella is suitable for use in mammals, humans, fish, birds and lower mammals.

U.S. Pat. No. 4,915,965 discloses a health food containing Dunaliella algae and a process for production of encapsulated foodstuff containing Dunaliella algae. Dried powder of Dunaliella algae is granulated together with other materials to make a granulation and the granulation is encapsulated in a hard capsule. Dried powder of Dunaliella algae is also suspended in an emulsifier along with other materials to form a suspension and the suspension is encapsulated in a soft capsule. The use of Chlorella in tablet form is disclosed. However, U.S. Pat. No. 4,915,965 does not disclose an encapsulated gel formulation of live algae, such as live Chlorella, Spirulina or Dunaliella. This patent teaches against use of living algae. Therefore, according to these teachings, an increase in energy content as a result of photysynthesis after encapsulation is infeasible.

U.S. Pat. No. 4,143,162 discloses an encapsulated foodstuff containing a powdered water extract of Chlorella algae. This background art reference differs from the present invention in that powdered water extracts of Chlorella algae are used and not live Chlorella algae. Again, this patent teaches against use of living algae. Therefore, according to these teachings, an increase in energy content as a result of photysynthesis after encapsulation is infeasible.

Japanese Patent No. 59-63146 discloses the use and preparation of encapsulated live algae as a fish food. However, this background art reference does not disclose use of encapsulated algae as a health food or supplement for other species or the use and preparation of fiber containing algae, as is disclosed in the present invention. Further, this patent does not teach encapsulation or entrapment of Dunaliella spp algae. Algae of the genus Dunaliella are characterized by an exceptionally fragile cell wall which requires the use of novel entrapment methods as disclosed in the instant application.

Japanese patent 60190465 teaches “. . . adding heterotrophic chlorella or its processed product to the pastry of the noodles”. This patent does not teach the use of Dunaliella. Further, these teachings are for heterotrophic algae, not autotrophic algae.

U.S. Pat. No. 4,744,996 teaches an algae based food. However, teachings of this patent require heating to 100 degrees centigrade. This heating precludes subsequent photosynthesis.

Japanese Patent 59-63146 teaches an animal feed containing algae, but does not teach the use of Dunaliella. As explained hereinabove, Dunaliella requires special care in order to preserve its cellular structure during processing. Such care is not taught in the prior art.

U.S. Pat. No. 4,380,551 teaches methods of producing foodstuff by matting seed. These teachings do not include encapsulation or entrapment of Dunaliella and other algae, a significant disadvantage.

Preparation and preservation of Chlorella, Dunaliella or Spirulina either in tablets, granules or in liquid extract may result in destruction of most of the physiologically active ingredients. None of the background art references has taught or suggested the encapsulation of living algae such as Chlorella, Dunaliella and Spirulina or others, in order to retain the effects of the active ingredients for use as a health food or supplement for mammals, humans, lower mammals or birds. Furthermore, none of these background art references has taught or suggested the use or preparation of living algae entrapped in elongated fibers of different lengths and widths.

There is thus a widely recognized need for, and it would be highly advantageous to have vegetarian foodstuff containing entrapped viable algae devoid of the above limitations.

### SUMMARY OF THE INVENTION

According to one aspect of the present invention there is provided a food composition for ingestion in a non-marine environment. The food composition includes primarily entrapped living algae cells, the entrapped living algae cells capable of photosynthetic multiplication; wherein the photosynthetic multiplication increases a nutritional content of the food composition.

According to another aspect of the present invention there is provided a food composition. The food composition includes primarily fibrous structures containing living algae cells. The living algae cells are capable of photosynthetic multiplication within the fibrous structures. The photosynthetic multiplication increases a nutritional content of the food composition.

According to yet another aspect of the present invention there is provided a method of encapsulating live algae cells in a food composition. The method includes the steps of. (a) suspending live algae cells in a suitable encapsulating medium to produce an algae mixture; (b) dropping drops of the algae mixture into a setting bath; (c) bubbling a gas non-reactive to the setting bath therethrough in order to reduce distortion and splashing of tie drops; and (d) incubating the drops in the setting bath to form encapsulated living algae cells in capsules.

According to still another aspect of the present invention there is provided a method of entrapping live algae cells in elongated edible strands, the method includes the steps of: (a) suspending live algae cells in a suitable entrapping medium to produce an algae mixture; (b) injecting the algae mixture into a setting bath to produce elongated fibrous structures; and (c) incubating the elongated fibrous structures in the setting bath to form the edible strands with live algae cells entrapped therein.

According to further features in preferred embodiments of the invention described below, the entrapped living algae cells include algae of the genus Dunaliella.

According to still her features in the described preferred embodiments the entrapped living algae cells include algae belonging to at least one additional genus.

According to still further features in the described preferred embodiments the at least one additional genus is selected from the group consisting of Spirulina and Chlorella.

According to still further features in the described preferred embodiments the composition includes at least two distinct phases.

According to still further features in the described preferred embodiments the entrapped living algae cells are encapsulated.

According to still further features in the described preferred embodiments the entrapped living algae cells are entrapped within elongated fibrous structures.

According to still further features in the described preferred embodiments the living algae cells are present in a concentration of from about 1,000 to about 100,000,000 living algae cells per ml.

According to still further features in the described preferred embodiments the composition further includes a food flavoring.

According to still further features in the described preferred embodiments the composition further includes a food coloring.

According to still further features in the described preferred embodiments the entrapped living algae is exposed to light for an interval of time.

According to still further features in the described preferred embodiments the entrapped living algae is incubated in darkness at 4° C. for a period of time.

According to still further features in the described preferred embodiments the entrapped living algae is incubated at a temperature of between about 4° C. to about 40° C.

According to still further features in the described preferred embodiments the step of suspending is accomplished using a low speed orbital shaker for 2 to 24 hours in order to prevent cell wall disruption.

According to still further features in the described preferred embodiments the step of dropping is accomplished using a force selected from the group consisting of gravity and an applied pressure of no more than 1 atmosphere.

According to still further features in the described preferred embodiments the step of dropping is accomplished using co-axial nozzles, such that the capsules resulting from the method are each comprised of at least one inner capsule and at least one outer capsule, the at least one inner and the at least one outer capsules having different compositions.

According to still further features in the described preferred embodiments the encapsulating medium includes a salt of alginic acid.

According to still further features in the described preferred embodiments the salt of alginic acid is sodium alginate.

According to still further features in the described preferred embodiments the step of incubating the encapsulated living algae cells at a temperature of between about 4° C. to about 40° C.

According to still further features in the described preferred embodiments the setting bat includes an edible water soluble calcium salt.

According to still further features in the described preferred embodiments the capsule is a semi-permeable capsule.

According to still further features in the described preferred embodiments the step of suspending is accomplished using a low speed orbital shaker for 2-24 hours in order to prevent cell wall disruption.

According to still further features in the described preferred embodiments the step of injecting is accomplished using a force selected from the group consisting of gravity and an applied pressure of no more than 1 atmosphere.

According to still further features in the described preferred embodiments the step of injecting is accomplished using co-axial nozzles, such that the elongated edible strands resulting from the method are each comprised of at least one inner strand and at least one outer stand, the at least one inner and the at least one outer strands having different compositions.

The present invention provides an entrapped health food containing viable unicellular algae, preferably Chlorella, Dunaliella and Spirulina. In particular it concerns a food composition for ingestion in a non-marine environment comprising encapsulated living algae cells. In addition the present invention provides a food composition comprising elongated fibers containing algae cells for use in mammals, humans, lower mammals, fish and birds. Algae cells are entrapped in capsules, or in spaghetti-like fiber form, of salts of alginic acid, such as calcium alginate or of alternative edible materials for entrapment The present invention provides a food where all the physiologically active ingredients are maintained during entrapment. Moreover, the entrapped health food of the present invention enables the algae cells to multiply inside the capsules or fibers and therefore result in an increased number of algae cells per capsule or fiber. The capacity of the algae to multiply is possible due to exposure to light, the expandable matrix of the capsule or fiber and flux of nutrition to the algae is possible through the semi-permeable matrix of the capsule or fiber.

According to the teachings of the present invention there is provided in a first embodiment a food composition for ingestion in a non-marine environment comprising entrapped living algae cells.

In a preferred embodiment the living algae cells include Chlorella.

In a preferred embodiment the living algae cells include Dunaliella.

In a preferred embodiment the living algae cells include Spirulina.

In a preferred embodiment the living algae cells include a mixture of Chlorella and Spirulina.

In a preferred embodiment the entrapped living algae cells are encapsulated.

In a preferred embodiment the entrapped living algae cells are elongated fiber containing algae cells.

In a preferred embodiment the living algae cells are present in a concentration of from about 1,000 to about 100,000,000 living algae cells per cm³ of capsule.

In a preferred embodiment the food composition further includes a food flavoring.

In a preferred embodiment the food composition further includes a food coloring.

In a preferred embodiment the entrapped living algae is kept under light for extended time intervals.

In a preferred embodiment the entrapped living algae is kept in the dark at 4° C. for a period of time.

In a preferred embodiment the food composition is suitable for mammals.

In a preferred embodiment the food composition is suitable for humans.

In a preferred embodiment the food composition is suitable for lower mammals.

In a preferred embodiment the food composition is suitable for birds.

In a preferred embodiment the food composition is used as a vegetarian food.

In a preferred embodiment the food composition is used as a food supplement for animals.

In a preferred embodiment the food composition is used for the treatment selected from the group consisting of stimulating the immune system, antioxidant and antitumour activity, anti-viral activity, anti-allergy properties, anti-aging properties, cold preventative action, restoring bowel regularity, normalizing bowel flora, stimulating repair of damaged bowel, detoxifying the body, post-operative aid, aiding children with growth problems, pregnancy, weight gain, weight loss, solid bone building, healthy gums, teeth or liver and combinations thereof.

In a preferred embodiment the treatment includes preventative treatment.

In a preferred embodiment the food composition is a vitamin supplement.

In a preferred embodiment the vitamin supplement is a vitamin B supplement.

In a preferred embodiment the vitamin B supplement is a vitamin B12 supplement,

In a preferred embodiment the vitamin supplement is a provitamin A supplement. in a preferred embodiment the food supplement is a chlorophyll supplement.

In a preferred embodiment the food composition is a β-carotene supplement.

In a preferred embodiment the food composition is a Chlorella Growth Factor supplement.

In a preferred embodiment the food composition is a mineral supplement.

In a second embodiment the present invention provides a food composition comprising fiber containing living algae cells.

In a preferred embodiment of the fiber containing living algae food composition living algae cells are selected from the group consisting of Chlorella, Dunaliella, Spirulina and mixtures of Chlorella and Spirulina.

In a preferred embodiment of the fiber containing living algae food composition living algae cells are present in a concentration of from about 1,000 to about 100,000,000 living algae cells per cm³ of capsule.

In a preferred embodiment the fiber containing living algae food composition further includes a food flavoring.

In a preferred embodiment the fiber containing living algae food composition further includes a food coloring.

In a preferred embodiment of the fiber containing living algae food composition entrapped living algae is kept under light for extended time intervals.

In a preferred embodiment of the fiber containing living algae food composition entrapped living algae is kept in the dark at 4° C. for a period of time.

In a preferred embodiment of the fiber containing living algae food composition the food composition is suitable for a subject selected from the group consisting of mammals, humans, lower mammals, fish and birds.

In a preferred embodiment of the fiber containing living algae food composition the food composition is used as a vegetarian food.

In a preferred embodiment of the fiber containing living algae food composition the food composition is used as a food supplement for animals.

In a preferred embodiment of the fiber containing living algae food composition the food composition is used for the treatment selected from the group consisting of stimulating the immune system, antioxidant and antitumour activity, anti-viral activity, anti-allergy properties, anti-aging properties, cold preventative action, restoring bowel regularity, normalizing bowel flora, stimulating repair of damaged bowel, detoxifying the body, post-operative aid, aiding children with growth problems, pregnancy, weight gain, weight loss, solid bone building, healthy gums, teeth or liver and combinations thereof.

In a preferred embodiment of the fiber containing living algae food composition the treatment includes preventative treatment.

In a preferred embodiment of the fiber containing living algae food composition the food composition is a vitamin supplement.

In a preferred embodiment of the fiber containing living algae food composition the vitamin supplement is selected from the group consisting of vitamin B supplement, vitamin D12 supplement and provitamin A supplement and combinations thereof.

In a preferred embodiment of the fiber containing living algae food composition the food supplement is selected from the group consisting of a chlorophyll supplement, β-carotene supplement, a Chlorella Growth Factor supplement and a mineral supplement and combinations thereof

In a third embodiment the present invention provides a method of encapsulating live algae cells in a food composition comprising the steps of: (a) suspending live algae cells in a suitable encapsulating medium to produce an algae mixture; (b) dropping drops of the algae mixture into a setting bath; (c) bubbling a gas non-reactive to the setting bath to reduce distortion and splashing of said drops; and (d) incubating the drops in a setting bath to form encapsulated living algae cells in capsules.

In a preferred embodiment of the method of encapsulating live algae cells the living algae cells include Chlorella.

In a preferred embodiment of the method of encapsulating live algae cells the living algae cells include Dunaliella.

In a preferred embodiment of the method of encapsulating live algae cells the living algae cells include Spirulina.

In a preferred embodiment of the method of encapsulating live algae cells the living algae cells include a mixture of Chlorella and Spirulina.

In a preferred embodiment of the method of encapsulating live algae cells the encapsulating medium includes a salt of alginic acid.

In a preferred embodiment of the method of encapsulating live algae cells the salt of alginic acid is sodium alginate.

In a preferred embodiment of the method of encapsulating live algae cells the encapsulated living algae cells are kept at a temperature of between about 4° C. to about 40° C.

In a preferred embodiment of the method of encapsulating live algae cells the encapsulated living algae cells are kept in light for extended time intervals.

In a preferred embodiment of the method of encapsulating live algae cells the encapsulated living algae are kept in the dark at 4° C. for a period of time.

In a preferred embodiment of the method of encapsulating live algae cells the setting bath includes an edible water soluble calcium salt.

In a preferred embodiment of the method of encapsulating live algae cells the capsule is a semi-permeable capsule.

In a fourth embodiment the present invention provides a method of entrapping live algae cells in an elongated fiber form in a food composition comprising the steps of: (a) suspending live algae cells in a suitable entrapping medium to produce an algae mixture; (b) injecting the algae mixture into a setting bath to produce elongated fibers; and (c) incubating the elongated fibers in a setting bath to form live algae cells entrapped in an elongated fiber.

In a preferred embodiment of the method of entrapping live algae cells in an elongated fiber form in a food composition the food composition is selected from the group consisting of a food composition for mammals, humans, lower mammals, birds and fish.

The term ‘non-marine environment’ as used herein refers to an environment where live fish are absent.

### DESCRIPTION OF THE PREFERRED EMBODIMENTS

The present invention is of a food composition for ingestion in a non-marine environment comprising entrapped living algae cells, preferably containing Chlorella, Dunaliella and Spirulina. In particular the present invention concerns algae cells entrapped in capsules, preferably semi-permeable capsules and more preferably capsules of salts of alginic acid, such as calcium alginate, or alternative edible materials for encapsulation for use as a food or health supplement in mammals, humans, lower mammals and birds.

In addition the present invention concerns algae cells entrapped in fiber, preferably elongated spaghetti-like fibers of salts of alginic acid or alternative edible materials for algae entrapment for use as a food or health supplement in mammals, humans, lower mammals, fish and birds.

The capsules or fibers can optionally contain food flavoring, food coloring and other additives. The capsules or fibers are kept under conditions of light, preferably sunlight or artificial light and at temperatures between from about 4° C. to temperatures, such as 40° C. in medium suitable for viable algae, such as medium containing NH₄NO₃, CaCl₂2H₂O, MgSO₄,7H₂O, K₂HPO₄, KH₂PO₄ and NaCl. It is possible to store the algae for variable time periods of at least but not limited to 3 months and preferably 1 month at 4° C. in the dark and for extended time periods of at least, but not limited to over one year, preferably for about 3 months under conditions of light. Under conditions of darkness, the algae do not multiply The addition of light causes the algae to photosynthesize and multiply. The methods of entrapment preserve the natural color of the algae. However, the capsules can be formed in different shapes and different colors. The method of the present invention can also be adopted to form capsules or fibers of different sizes, depending on the intended use of the product. Additives, such as food flavorings, food colorants, and preservatives can optionally be added.

The present invention provides a food where all the physiologically active ingredients are maintained during entrapment in encapsulation or in fiber form. Moreover, the entrapped health food of the present invention enables the algae cells to multiply inside the capsules or fibers and therefore result in an increased number of algae cells per capsule or fiber. In this way the entrapped algae has a higher nutritional content. The food composition of the present invention, will therefore be significantly richer in the physiologically active ingredients contained in the algae than algae containing food products of the background art.

The encapsulated algae can be ingested by mammals, humans, lower mammals and birds. The elongated fiber containing algae is suitable for use in mammals, humans, lower mammals, fish and birds.

The present invention also provides methods of entrapment of the living algae cells to produce entrapped living algae for use as a health and food supplement.

Fresh algae are used as the starting material for the encapsulated product of the present invention. Living unicellular algae are suspended in a suitable medium such as water, containing various salts for conservation of cell function. The living algae are mixed with edible alginate salts of alginic acid, such as about 0.5 to about 10% sodium alginate or alternative edible materials for encapsulation, Drops of this mixture are dropped from about 2 to about 30 cm into a setting bath containing an edible water-soluble calcium salt, such as calcium chloride, or calcium lactate. The drops (approximately 0.1 mm to about 20 mm) are left in the bath for a period of between about 1 to about 30 minutes, after which time the capsules become firm and are easily handled without breaking. Capsules are then removed from the bath and washed. The encapsulated algae product is stored in containers in fresh water medium containing various salts, such as NH₄.NO₃, CaCl₂.2H₂O, MgSO₄.7H₂O, K₂HPO₄, KH₂PO₄ and NaCl. The algae are kept under sunlight or artificial light at between about 4 to about 40° C. for various time periods, and alternatively the algae can be kept at 4° C. with no light. The living algae cells are typically present in a concentration of from about 1,000 to about 100,000,000 living algae cells per cm³ of capsule, however higher and lower concentrations are possible.

In an alternative method of encapsulation of the live algae an aqueous solution of sodium alginate or alternative edible materials for encapsulation is co-extruded with living algae to form algae drops coated with alginate solution. The coated drops are then dropped from 2 to about 30 cm into an aqueous setting bath containing as setting agent an edible, water-soluble calcium salt to set the solution to a gel, such as calcium alginate and thereby forms encapsulated algae. Distortion of the drops on entering the setting bath and disruptive splashing of the bath are minimised by bubbling a gas that is non-reactive to the setting bath. The gas forms a foam at the surface of the setting bath so that distortion of the drops and splashing of the alginate solution and algae is reduced.

Fresh algae cells are used as the starting material for the spaghetti-like fiber product of the present invention. Living unicellular algae are suspended in a suitable medium such as water, containing various salts for conservation of cell function. The living algae are mixed with edible alginate salts of alginic acid, such as about 0.5 to about 10% sodium alginate or alternative edible materials for algae entrapment. The mixture is injected through a nozzle(s) or needle(s) preferably of about, but not limited to about 1 to about 10 mm in diameter, into a setting bath containing calcium chloride or calcium lactate. Fibers are left in the bath for a period of between about 1 to about 30 minutes, after which time the fibers become firm and are easily handled without breaking. Fibers are then removed from the bath and washed. The spaghetti-like fibers containing algae are stored in containers in fresh water medium containing various salts, such as NH₄.NO₃, CaCl₂.2H₂O, MgSO₄.7H₂O, K₂HPO₄, KH₂PO and NaCl. The algae are kept under sunlight or artificial light at between about 4 to about 40° C. for various time periods, or the algae can be kept at 4° C. with no light. The living algae cells are typically present in a concentration of from about 1,000 to about 100,000,000 living algae cells per cm³ of fiber, however higher and lower concentrations are possible. The fiber containing algae can have different lengths, widths and shapes.

The food composition of the present invention has many uses. It can be used as a vegetarian type food, preferably as a vegetarian type caviar or pasta for human use. Alternatively, the encapsulated live algae or spaghetti-like fibers containing algae of the present invention can be used as a food supplement for animals.

Additionally, the entrapped live algae can be used as a health supplement Chlorella has been disclosed to have therapeutic effects in stimulating the immune system, antioxidant and antitumour activity, anti-aging properties, cold preventative action, restoring bowel regularity, normalizing bowel flora, stimulating repair of damaged bowel, detoxifying the body, post-operative aid, aiding children with growth problems, pregnancy, weight gain, weight loss, solid bone building, healthy gums, teeth or liver. The food composition of the present invention can therefore be used for treatment of the aforementioned conditions, or as a preventative treatment.

Algae contain various physiologically active ingredients. Chlorella, Dunaliella and Spirulina are known in the background art to be rich in more than 20 different vitamins, amino acids and minerals, are abundant in beta-carotene and chlorophyll, as well as growth factor. The food composition of the present invention can therefore be used as a supplement of these physiologically active ingredients, such as a vitamin B12 supplement, a chlorophyll supplement and a P-carotene supplement.

According to one aspect of the present invention there is provided a food composition for ingestion in a non-marine environment, The food composition includes primarily entrapped living algae cells. The entrapped living algae cells are capable of photosynthetic multiplication. This photosynthetic multiplication increases a nutritional content of the food composition. Therefore, the nutritional content, for example the emergy content, of the food composition may increase during storage if light is present in the storage area. Preferably, the entrapped living algae cells include algae of the genus Dunalliela. More preferably, the entrapped living algae cells include algae belonging to at least one additional genus. Most preferably, the additional genus includes Spirulina or Chlorella.

In some casts, it can be advantageous for the composition to include at least two distinct phases. For example, an inner static phase consisting of an algae product (e.g. dry algae powder 0.5-10% and/or disrupted algae cell particles) entrapped in a non transparent edible matrix such as gelatin or alginate, and an autotrophic/dynamic phase containing viable algae cells in a transparent edible matrix such as alginate.

According to various preferred embodiments the entrapped living algae cells are either encapsulatedor are entrapped within elongated fibrous structures. Preferably the living algae cells are present in a concentration of from about 1,000 to about 100,000,000 living algae cells per ml. of finished composition. In order to increase palatability to humans, the composition may further include a food flavoring such as, for example, a fish oil, a spice, salt, vegetable powder or a meat extract. In order to increase consumer acce[patability, the composition may further include a food coloring to mask the color of the algae which may be unappealing.

According to various embodiments of the invention, the entrapped living algae may exposed to light for an interval of time. This is done to allow photosynthetic processes to occur, thereby maintaining, or even eicreasing, the nutritional content of the composition. Alternately, or additionally, the entrapped living algae is incubated in darkness at 4° C. for a period of time. This may be done to preserve the composition once a desired stage of maturation is achieved. Alternately, or additionally, the entrapped living algae is incubated at a temperature of between about 4° C. to about 40° C. in order to control the metabolic rate thereof.

According to another aspect of the present invention there is provided a food composition. The food composition includes primarily fibrous structures containing living algae cells. The living algae cells are capable of photosynthetic multiplication within the fibrous structures. The photosynthetic multiplication increases a nutritional content of the food composition.

According to yet another aspect of the present invention there is provided a method of encapsulating live algae cells in a food composition. The method includes the step of suspending live algae cells in a suitable encapsulating medium to produce an algae mixture. The method further includes the steps of dropping drops of the algae mixture into a setting bath and bubbling a gas non-reactive to the setting bath therethrough. The bubbles reduce distortion and splashing of the drops. The method further includes the step of incubating the drops in the setting bath to form encapsulated living algae cells in capsules.

The step of suspending may be accomplished, for example, using a low speed orbital shaker in order to prevent cell wall disruption. Depending on the exact shaker employed, 2 to 24 hours may be required to assured good suspension with maximum cell viability. The step of dropping may be accomplished using for example, the force of gravity or an applied pressure of about 1 atmosphere or less.

According to some embodiments of the invention the step of dropping is accomplished using co-axial nozzles. In this case, the capsules resulting from the method each include of at least one inner capsule and at least one outer capsule. Preferably, the at least one inner and the at least one outer capsule each have different compositions. The encapsulating medium may include, for example, a salt of alginic acid such as sodium alginate, calcium alginate or potassium alginate. The setting bath may include an edible water soluble calcium salt. Preferably, the resultant capsule is a semi-permeable capsule.

According to still another aspect of the present invention there is provided a method of entrapping live algae cells in elongated edible strands, the method includes the step of suspending live algae cells in a suitable entrapping medium to produce an algae mixture as described hereinabove. The method further includes the steps of injecting the algae mixture into a setting bath to produce elongated fibrous structures and incubating the elongated fibrous structures in the setting bath to form the edible strands with live algae cells entrapped therein.

The step of injecting may be accomplished, for example, using the force of gravity or an applied pressure of about I atmosphere or less. In order to produce a two phase strand, the step of injecting may be accomplished using co-axial nozzles. In this way the elongated edible strands resulting from the method each include at least one inner strand and at least one outer strand the at least one inner and the at least one outer strands having different compositions.

It is to be understood that the invention is not limited in its application to the details of construction and the arrangement of the components set forth in the following description. The invention includes other embodiments and can be practiced or implemented in various ways. Also it is to be understood that the phraseology and terminology employed herein is for the purpose of description only and should not be regarded as limiting.

The present invention may be better understood with reference to the examples and the accompanying description.

Example 1: Preparation of encapsulated Chlorella for use as a food supplement for mammals, humans and lower mammals

Living unicellular Chlorella algae were suspended in a suitable medium such as water, containing various salts. The medium contained NH₄.NO₃ (0.125 g/L), CaCl₂.2H₂O (0.025 g/L), MgSO₄.7H₂O (0.075 g/L), K₂HPO₄ (0.075 g/L), KH₂PO₄ (0.175 g/L) and NaCl (0.025 g/L) and living algae mixed with edible salts of alginic acid (approximately 1/1, w/v), such as 0.5-10% sodium alginate.

Drops of this mixture were dropped from about 2-30 cm into a setting bath containing water (approximately 97%) and an edible water soluble calcium salt (approximately 3%), such as calcium chloride or calcium lactate. The drops (approximately 0.1 mm-20 mm) were left in the bath for a period of between about 1-30 minutes after which time the capsules became firm and were easily handled without breaking. Capsules were then removed from the bath and washed. The encapsulated algae product was stored in glass or plastic containers with fresh water medium containing various salts. The algae were kept under sunlight or artificial light at between 4-40° C., or alternatively at 4° C. in the dark for various time periods of storage.

Example 2: Preparation of encapsulated Dunaliella for use as a food supplement for mammals humans and lower mammals

Living unicellular Dunaliella algae were suspended in a suitable medium such as about 100 ml seawater, containing various salts. The medium contained NaCl (23 g), MgCl, (5 g), Na₂SO₄ (4 g) and living algae mixed with edible salts of alginic acid (approximately 1/1, w/v), such as 0.5-10% sodium alginate.

Drops of this mixture were dropped from about 2-30 cm into a setting bath containing water (approximately 98%) and an edible water soluble calcium salt (approximately 2%), such as calcium chloride or calcium lactate. The drops (approximately 0.1 mm-20 mm) were left in the bath for a period of between about 1-30 minutes after which time the capsules became firm and were easily handled without breaking. Capsules were then removed from the bath and washed. The encapsulated Dunaliella product was stored in glass or plastic containers with fresh water medium containing various salts. The encapsulated Dunaliella algae were kept under sunlight or artificial light at between 4-40° C., or alternatively at 4° C. in the dark for various time periods of storage.

Example 3: Preparation of eucapsulated Spirulina for use as a food supplement for mammals, humans and lower mammals

Living unicellular Spirulina algae were suspended in a suitable medium such as water containing various salts. The medium contained NaHCO₃ (13.6 g/L), Na₂CO₃ (4.0 g/L), NH₄.NO₃ (1.25 g/L), K₂SO₄ (1.0 g/L), CaCl₂.2H₂O (0.04 g/L), MgSO₄.7H₂O (0.2 g/L), K₂HPO₄ (0.5 g/L), and NaCl (1.0 g/L)and trace metal elements such as, FeCl₃.6H₂O (0.5 mg/L), MnCl₂.4H₂O (0.25 mg/L), ZnCl₂ (0.025 mg/L) and CuSO₄.5H₂O (0.020 mg/L) and living algae mixed with edible salts of alginic acid (approximately 1/1, w/v), such as 0.5-10% sodium alginate.

Drops of this mixture were dropped from about 2-30 cm into a setting bath containing water (approximately 98%) and an edible water soluble calcium salt (approximately 2%), such as calcium chloride or calcium lactate. The drops (approximately 0.1 mm -20 mm) were left in the bath for a period of between about 1-30 minutes after which time the capsules became firm and were easily handled without breaking. Capsules were then removed from the bath and washed. The encapsulated Spirulina product was stored in glass or plastic containers with fresh water medium containing various salts. The encapsulated Spirulina algae were kept under sunlight or artificial light at between 4-40° C., or alternatively at 4° C. in the dark for various time periods of storage.

Example 4: Preparation of encapsulated mixture of Chlorella and Spirulina for use as a food supplement for mammals, humans and lower mammals

A mixture of living unicellular Chlorella algae and Spirulina algae are suspended in a suitable medium such as water, containing various salts. The medium contains NH₄.NO₃ (0.125 g/L), CaCl₂.2H₂O (0.025 g/L), MgSO₄.7H₂O (0.075 g/L), K₂HPO₄ (0.075 g/L), KH₂PO₄ (0.175 g/L) and NaCl (0.025 g/L) and living algae mixed with edible salts of alginic acid (approximately 1/1, w/v), such as 0.5-10% sodium alginate.

Drops of this mixture are dropped from about 2-30 cm into a setting bath containing water (approximately 98%) and an edible water soluble calcium salt (approximately 2%), such as calcium chloride or calcium lactate. The drops (approximately 0.1 mm-20 mm) are left in the bath for a period of between about 1-30 minutes after which time the capsules become firm and are easily handled without breaking. Capsules are then removed from the bath and washed. The encapsulated mixture of Chlorella and Spirulina algae are stored in glass or plastic containers with fresh water medium containing various salts. The encapsulated Chlorella and Spirulina algae are kept under sunlight or artificial light at between 4-40° C., or alternatively at 4° C. in the dark for various time periods of storage.

Example 5: Alternative method of encapsulating algae

An aqueous solution of alginate is co-extruded with living algae to form algae drops coated with alginate solution. The coated drops are then dropped from about 2 to 30 cm into an aqueous setting bath containing as setting agent an edible, water-soluble calcium salt to set the solution to a gel, such as calcium alginate and thereby forms encapsulated algae. Distortion of the drops on entering the setting bath and disruptive splashing of the bath are minimised by bubbling a gas that is non-reactive to the setting bath. The gas forms a foam at the surface of the setting bath so that distortion of the drops and splashing of the alginate solution and algae is reduced.

Example 6: Method of preparation of spaghetti-like fiber containing Chlorella

Living unicellular Chlorella were suspended in a suitable medium such as water, containing various salts. The medium contained NH₄.NO₃ (0.125 g/L), CaCl₂.2H₂O (0.025 g/L), MgSO₄.7H₂O (0.075 g/L), K₂HPO₄ (0.075 g/L), KH₂PO₄ (0.175 g/L) and NaCl (0.025 g/L) and living algae mixed with edible salts of alginic acid (approximately 1/1, w/v), such as 0.5-10% sodium alginate.

The mixture was injected through a needle into a setting bath containing water (approximately 98%) and an edible water soluble calcium salt (approximately 2%), such as calcium chloride or calcium lactate. The fibers (approximately 1-10 mm in diameter) were left in the bath for a period of between about 1-30 minutes after which time the fibers became firm and were easily handled without breaking. Fibers were then removed from the bath and washed. The spaghetti-like fibers containing Chlorella product was stored in glass or plastic containers with fresh water medium containing various salts. The spaghetti-like elongated fibers containing Chlorella product were kept under sunlight or artificial light at between 4-40° C., or alternatively at 4° C. in the dark for various time periods of storage.

Example 7: Method of preparation of spaghetti-like fiber containing Dunaliella

Living unicellular Dunaliella were suspended in a suitable medium such as about 1000 ml seawater, containing various salts. The medium contained NaCl (23 g), MgCl2 (5 g), Na₂SO₄ (4 g) and living algae mixed with edible salts of alginic acid (approximately 1/1, w/v). such as 0.5-10% sodium alginate.

The mixture was injected through a needle into a setting bath containing water (approximately 98%) and an edible water soluble calcium salt (approximately 2%), such as calcium chloride or calcium lactate. The fibers (approximately 1-10 mm in diameter) were left in the bath for a period of between about 1-30 minutes after which time the fibers became firm and were easily bandied without breaking. Fibers were then removed from the bath and washed. The spaghetti-like fibers containing Dunaliella product was stored in glass or plastic containers with fresh water medium containing various salts. The spaghetti-like elongated fibers containing Dunaliella product were kept under sunlight or artificial light at between 4-40° C., or alternatively at 4° C. in the dark for various time periods of storage,

Example 8: Method of preparation of spaghetti-like fiber containing Spirulina

Living unicellular Spirulina were suspended in a suitable medium such as water, containing various salts. The medium contained NaHCO₃ (13.6 g/L), Na₂CO₃ (4.0 g/L), NH₄.NO₃ (1.25 g/L), K₂SO₄ (1.0 g/L), CaCl₂.2H₂O (0.04 g/L), MgSO₄.7H₂O (0.2 g/L), K₂RPO₄ (0.5 g/L), and NaCl (1.0 g/L)and trace metal elements such as, FeCl₃.6H₂O (0.5 mg/L), MnCl₂.4H₂O (0.25 mg/L) ZnCl₂ (0.025 mg/L) and CuSO₄.5H₂O (0.020 mg/L) and living algae mixed with edible salts of alginic acid (approximately 1/1, w/v), such as 0.5-10% sodium alginate.

The mixture was injected through a needle into a setting bath containing water (approximately 98%) and an edible water soluble calcium salt (approximately 2%), such as calcium chloride or calcium lactate. The fibers (approximately 1-10 mm in diameter) were left in the bath for a period of between about 1-30 minutes after which time the fibers became firm and were easily handled without breaking. Fibers were then removed from the bath and washed. The spaghetti-like fibers containing Spirulina product was stored in glass or plastic containers with fresh water medium containing various salts. The spaghetti-like elongated fibers containing Spirulina product were kept under sunlight or artificial light at between 4-40° C., or alternatively at 4° C. in the dark for various time periods of storage.

Example 9: Method of use of encapsulated algae or spaghetti-like fibre containing algae as a vegetarian food

The encapsulated living algae or fibers containing algae such as Chlorella or Dunaliella or Spirolina were used as a vegetarian food. Chlorella, Dunaliella and Spirulina are extremely nutritious,. being rich sources of protein and carbohydrate. Entrapped live algae is useful as a vegetarian type caviar or pasta depending on the entrapment process. A typical serving is from about 1-20 g, although smaller and larger doses are possible.

The encapsulated algae or fiber containing algae has an algae like taste, but can be made optionally sweet or salty using different additives. Alternatively, different additives can be added to produce different flavors, to increase storage and/or freezability. Furthermore, the entrapment of the live algae preserves the natural color of the algae.

Example 10: Method of use of encapsulated algae or spaghetti-like fibers containing algae for preventative and therapeutic means

The encapsulated algae or fibers containing algae of the present invention can be used as a health supplement. Chlorella and Spirulina stimulate the immune system, display antioxidant and antitumour activity, exhibit anti-aging properties, have cold preventative action, restore bowel regularity, normalize bowel flora and stimulate repair of damaged bowel. Chlorella and Spirulina have also been reported to detoxify the body, be useful for post-operative patients, older people, children with growth problems and pregnant women, and is useful in weight gain and weight loss In addition, Chlorella and Spirulina have been shown to promote solid bone building, healthy gums, teeth and liver.

Dunaliella algae have antioxidant properties and the ability to reduce virus and allergy symptoms.

The encapsulated algae or fiber containing algae can be taken as a preventative measure or used for the rapeutic activity in any of the aforementioned indications.

Dosing is dependent on the responsiveness of the subject to the encapsulated or fiber containing algae. Preferably the dose is from about 1 to about 20 g, however higher or lower doses are possible. The amount received by the subject is controlled. For example, the dose and frequency of dosing would be dependent on the responsiveness of the subject. Persons of ordinary skill in the art can easily determine optimum dosages, dosing methodologies and repetition rates.

Example 11: Method of use of entrapped living algae as a health supplement

Chlorella and Spirulina are rich in high quality proteins (50-60% of total mass), carbohydrate (15-20%), fat (10-15%), minerals (6%) and 4% moisture. Chlorella and Spirulina are one of the highest sources of chlorophyll and also contain Chlorella Growth factor and Spirulina Growth factor, and a higher concentration of Vitamin B12 than beef liver. The encapsulated or fiber containing Chlorella or Spirulina can be taken as a supplement of Vitamin B12, chlorophyll and growth factor or any of the other nutritional constituents contained in the algae. Additionally the entrapped algae can be taken as a health supplement for the combination effect of all the constituents of Chlorella or Spirulina.

Dunaliella algae contain proteins, lipids, sugars and minerals such as iron and calcium Dunaliella also contains vitamins such as provitamin A and vitamin B group compounds and a variety of other physiologically active ingredients, especially β-carotene. The encapsulated or fiber containing Dunaliella can be taken as a supplement of provitamin A and vitamin B group compounds and as a β-carotene supplement or as a supplement of any of the other nutritional constituents contained in the algae. Additionally the entrapped Dunaliella can be taken as a health supplement for the combination effect of all the constituents of Dunaliella.

Dosing is dependent on the responsiveness of the subject to the encapsulated algae. Preferably the dose is from about 1 to about 20 g, however higher or lower doses are possible. The amount received by the subject is controlled. For example, the dose and frequency of dosing would be dependent on the responsiveness of the subject. Persons of ordinary skill in the art can easily determine optimum dosages, dosing methodologies and repetition rates.

Example 12: Method of use of entrapped algae as a food supplement for animals

The encapsulated or fiber containing live algae is optionally useful as a food supplement for animals, in a non-marine environment. The encapsulated or fiber containing algae is highly nutritious and is a suitable alternative to available animal foods. Any amount of the encapsulated or fiber containing algae can be optionally used, however a typical amount is calculated depending on the size of the animal.

Example 13: Method of use of fiber containing algae as a fish food

The fiber containing live algae was used as a food supplement for fish. The fiber containing algae is highly nutritious and is a suitable alternative to available fish foods, The fiber containing algae without wishing to be limited by a single mechanism is hypothesized as being easier to see by the fish than encapsulated algae. Any amount of the fiber containing algae may be used, however a typical amount would be calculated depending on the size of the fish.

Example 14: Production of a multiphase composition

Any of Examples 1-8 may be accomplished using co-axial nozzles for dropping/injecting the solution into the setting bath. Typically, it is desirable to have each nozzle dispense a different suspension. For example, the inner nozzle may dispense an algae product entrapped in non transparent edible matrix such as gelatin or alginate. The algae product may include, for example, dry algae powder and/or disrupted algae cell particles. The outer nozzle may dispense entire living algae cells in a solution of transparent edible matrix such as alginate. Thus, nested capsules or nested fibers will form in the setting bath.

It will be appreciated that the above examples and descriptions are intended only to serve as examples, and that many other embodiments are possible within the spirit and the scope of the present invention.

What is claimed is:

1. A food composition for ingestion in a non-marine environment, the food composition comprising primarily entrapped living algae cells, said entrapped living algae cells capable of photosynthetic multiplication; wherein said photosynthetic multiplication increases a nutritional content of the food composition.

2. The composition of claim 1, wherein said entrapped living algae cells include algae of the genus Dunalliela.

3. The composition of claim 2 wherein said entrapped living algae cells include algae belonging to at least one additional genus.

4. The composition of claim 3, wherein said at least one additional genus is selected from the group consisting of Spirulina and Chlorella.

5. The composition of claim 1, wherein the composition comprises at least two distinct phases.

6. The food composition of claim 1, wherein said entrapped living algae cells are encapsulated.

7. The food composition of claim 1, wherein said entrapped living algae cells are entrapped within elongated fibrous structures.

8. The food composition of claim 1, wherein said living algae cells are present in a concentration of from about 1,000 to about 100,000,000 living algae cells per ml.

9. The food composition of claim 1, further comprising a food flavoring.

10. The food composition of claim 1, further comprising a food coloring.

11. The food composition of claim 1, wherein said entrapped living algae is exposed to light for an interval of time.

12. The food composition of claim 1, wherein said entrapped living algae is incubated in darkness at 4° C. for a period of time.

13. A food composition, the food composition comprising primarily fibrous structures containing living algae cells, said living alga cells capable of photosynthetic multiplication within said fibrous structures;
wherein said photosynthetic multiplication increases a nutritional content of the food composition.

14. The composition of claim 13, wherein said living algae cells include algae of the genus Dunalliela.

15. The composition of claim 14, wherein said entrapped living algae cells include algae belonging to at least one additional genus.

16. The composition of claim 15, wherein said at least one additional genus is selected from the group consisting of Spirulina and Chlorella.

17. The composition of claim 13, wherein the composition comprises at least two distinct phases.

18. The food composition of claim 13, wherein said living algae cells are present in a concentration of from about 1,000 to about 100,000,000 living algae cells per ml. of capsule.

19. The food composition of claim 13, wherein said entrapped living algae is exposed to light for an interval of time.

20. The food composition of claim 13, wherein said entrapped living algae is incubated at a temperature of between about 4° C. to about 40° C.

21. The food composition of claim 13, wherein said entrapped living algae is incubated in darkness at 4° C. for a period of time.

22. A method of encapsulating live algae cells in a food composition, the method comprising the steps of.
(a) suspending live algae cells in a suitable encapsulating medium to produce an algae mixture:
(b) dropping drops of said algae mixture into a setting bath;
(c) bubbling a gas nonreactive to said setting bath therethrough in order to reduce distortion and splashing of said drops; and
(d) incubating said drops in said setting bath to form encapsulated living algae cells in capsules.

23. The method of claim 22, wherein said step of suspending is accomplished using a low speed orbital shaker for 2 to 24 hours in order to prevent cell wall disruption.

24. The method of claim 22, wherein said step of dropping is accomplished using a force selected from the group consisting of gravity and an applied pressure of no more than 1 atmosphere.

25. The method of claim 22, wherein said step of dropping is accomplished using co-axial nozzles, such that said capsules resulting from the method are each comprised of at least one inner capsule and at least one outer capsule, said at least one inner and said at least one outer capsules having different compositions.

26. The method of claim 22, wherein said entrapped living algae cells include algae of the genus Dunalliela.

27. The method of claim 26, wherein said entrapped living algae cells include algae belonging to at least one additional genus.

28. The method of claim 27, wherein said at least one additional genus is selected from the group consisting of Spirulina and Chlorella.

29. The method of claim 22, wherein said encapsulating medium includes a salt of alginic acid.

30. The method of claim 29, wherein said salt of alginic acid is sodium alginate.

31. The method of claim 22, further comprising the step of incubating said encapsulated living algae cells at a temperature of between about 4° C. to about 40° C.

32. The method of claim 22, wherein said encapsulated living algae cells are exposed to light for an interval of time.

33. The method of claim 22, wherein said encapsulated living algae are incubated in the dark at 4° C. a period of time.

34. The method of claim 22 wherein said setting bath comprises an edible water soluble calcium salt.

35. The method of claim 22, wherein said capsule is a semi-permeable capsule.

36. A method of entrapping live algae cells in elongated edible strands, the method comprising the steps of:
(a) suspending live algae cells in a suitable entrapping medium to produce an algae mixture;
(b) injecting said algae mixture into a setting bath to produce elongated fibrous structures; and
(c) incubating said elongated fibrous structures in said setting bath to form the edible strands with live algae cells entrapped therein.

37. The method of claim 36, wherein said step of suspending is accomplished using a low speed orbital shaker for 2-24 hours in order to prevent cell wall disruption.

38. The method of claim 36, wherein said step of injecting is accomplished using a force selected from the group consisting of gravity and an applied pressure of no more than 1 atmosphere.

39. The method of claim 36, wherein said live algae cells entrapped therein include algae of the genus Dunalliela.

40. The method of claim 39, wherein said live algae cells entrapped therein include algae belonging to at least one additional genus.

41. The method of claim 40, wherein said at least one additional genus is selected from the group consisting of Spirulina and Chlorella.

42. The method of claim 36, wherein said step of injecting is accomplished using co-axial nozzles, such that the elongated edible strands resulting from the method are each comprised of at least one inner strand and at least one outer strand, said at least one inner and said at least one outer strands having different compositions.
